(19) United States
(12) Patent Application Publication
(10) Pub. No.: US 2020/0143946 A1
(43) Pub. Date: May 7, 2020

(54) PATIENT RISK SCORING AND EVALUATION SYSTEMS AND METHODS

(71) Applicant: South Side Master LLC, Houston, TX (US)

(72) Inventor: Russell F. LEWIS, Houston, TX (US)

(73) Assignee: South Side Master LLC, Houston, TX (US)

(21) Appl. No.: 16/673,726

(22) Filed: Nov. 4, 2019

Related U.S. Application Data
(60) Provisional application No. 62/756,005, filed on Nov. 5, 2018.

(51) Int. Cl. G16H 50/30; G16H 20/10; G16H 20/70; G16H 10/60; G16H 10/20

(52) U.S. Cl. CPC G16H 50/30; G16H 20/10; G16H 10/20; G16H 10/60; G16H 20/70

(57) ABSTRACT

Systems and methods for increasing throughput of a case management system include receiving data associated with a user, determining predicted risk factors based on the user data, calculating a risk score based on the predicted risk factors, and administering a service that validates the predicted risk factors. The service includes a dynamically-generated questionnaire having an initial question associated with a prioritized predicted risk and a plurality of subsequent questions that are generated based on previous answers and an optimization function that minimizes a total number of questions needed for the service to validate the predicted risk factors. Systems and methods further include administering risk mitigation services corresponding to validated risk factors, updating the user data with data collected from administered services, and updating the risk score based on the updated data. Systems and methods can include updating the risk score to reflect determined non-risk factors.

This application claims priority to U.S. provisional application 62/756,005 filed Nov. 5, 2018, the disclosures of which are herein incorporated by reference in their entirety.

### FIELD

### Background

It is well known that proper adherence to medical treatments is essential to achieving quality healthcare outcomes in patients. However, several challenges exist. Current platforms, such as the risk management platform described in US 2017/0357771 to Connolly et al., are directed to addressing the challenges by determining, based on various types of information, risk scores and risk factors for a patient, and then based on the determined risk factors, providing health planning recommendations. However, the accuracy and efficiency of such systems in determining the risk scores, risk factors, and recommendations is limited. For example, recommendations may be provided for risk factors that are not actual risk factors. Patients often become fatigued with traditional questionnaires provided by such management systems because they are long and numerous, thereby compromising the system's overall operational efficiency and effectiveness.

### SUMMARY

Below, various embodiments of the present invention are described for providing distribution systems and methods for medical articles.

In some embodiments, a method is provided, such as a method for increasing throughput of a case management system. The method includes receiving user data associated with a user, determining a plurality of predicted risk factors based on the user data, whereby each of the plurality of predicted risk factors having a weighted value, and calculating a first risk score based on the weighted values of the plurality of predicted risk factors. The method includes administering a question-based service that determines whether the plurality of predicted risk factors include a validated risk factor, the question-based service including a dynamically-generated questionnaire that has an initial question associated with a prioritized predicted risk factor of the plurality of predicted risk factors, and a plurality of subsequent questions that are each generated based on answers received to preceding questions and an optimization function that minimizes a total number of questions needed for the question-based service to determine whether the plurality of predicted risk factors include the validated risk factor. The method further includes, in accordance with a determination from the question-based service that the plurality of predicted risk factors include the validated risk factor: administering, to the user, a risk mitigation service corresponding to the validated risk factor identified by the question-based service, updating the user data with data collected from the administered risk mitigation service, and calculating a second risk score based on the updated user data. The method includes, in accordance with a determination from the question-based service that the plurality of predicted risk factors do not include validated risk factors, updating the first risk score to reflect that the plurality of predicted risk factors are non-risk factors.

It is noted that validating predicted risk factors via the dynamically-generated and optimized question-based service enhances operability of the system and makes the system more efficient (e.g., by determining and administering services for the risk factors when they are validated, reducing the number of inputs needed to perform the operation, reducing mistakes when operating/interacting with the device by way of its optimized questionnaire) which, additionally, reduces power and memory usage by enabling the system to be used more quickly, efficiently, and effectively.

Various embodiment and examples can be contemplated. In some examples, the dynamically-generated questionnaire is generated based on a preset time limit, whereby the total number of questions is constrained to a maximum number of questions allowable within the preset time limit.

In some examples, administering the question-based services includes: administering the initial question to the user, the initial question is associated with a plurality of predetermined allowable responses, analyzing, in real-time, an initial answer received from the user in response to the initial question by matching the initial answer to one of the plurality of predetermined allowable responses, determining whether the prioritized predicted risk factor is a validated risk factor or a non-risk factor based on the matched allowable response, and generating a next question of the plurality of subsequent questions based on the matched allowable response in real-time. In some examples, the method includes, in accordance with a determination that the prioritized predicted risk factor is the validated risk factor or the non-risk factor, generating the next question associated with another predicted risk factor, and in accordance with a determination that the prioritized predicted risk factor is not the validated risk factor or the non-risk factor, generating the next question associated with the same prioritized predicted risk factor. In some examples, the method includes administering the next question to the user, whereby the next question is associated with another plurality of predetermined allowable responses for categorizing, in real-time, a next answer received from the user in response to the next question.

In some examples, each of the plurality of subsequent questions is administered sequentially during the question-based service and the dynamically-generated questionnaire is updated in real-time in accordance with each answer received from the user.

In some examples, generating or updating the dynamically-generated questionnaire includes selecting questions from a questions database, whereby each question corresponds to a predicted risk factor and is associated with a set of predetermined allowable responses for validating the predicted risk factor, and each question is selected based on each previously matched allowable response.

In some examples, the method includes, while administering the question-based service, updating in real-time the first risk score in response to an answer received from the user to the dynamically-generated questionnaire, and generating or updating the dynamically-generated questionnaire based on the updated first risk score.

In some examples, the dynamically-generated questionnaire is generated based on a robustness function that minimizes errors in validating the predicted risk factors.

In some examples, at least one of the plurality of predicted risk factors was previously determined to be a mitigated risk factor or a non-risk factor for the user.

In some examples, the weighted value of each of the plurality of predicted risk factors for the user is based on a type of prescribed treatment for the user.

In some examples, the method includes, evaluating the data collected from the risk mitigation service while administering the risk mitigation service, determining whether the validated risk factor is a mitigated risk factor based on the data collected from the risk mitigation service, and in accordance with a determination that the validated risk factor is a mitigated risk factor: calculating the second risk score based on the updated user data, whereby the second risk score is lower than the first risk score, and in accordance with a determination that the validated risk factor is not a mitigated risk factor: determining a second risk mitigation service associated with the validated risk factor.

In some examples, the method includes, in accordance with a determination that the plurality of predicted risk factors include a plurality of validated risk factors: identifying a plurality of risk mitigation services, each risk mitigation service associated with each validated risk factor, prioritizing the plurality of risk mitigation services relative to one another to generate an intervention plan comprising a timeline for administering the plurality of risk mitigation services, and administering the plurality of services to the user in accordance with the intervention plan.

In some examples, the risk mitigation service includes at least one of providing an enrollment service to validate or invalidate multiple risk factors, providing access to financial assistance, providing disease or drug related education services, providing transportation services, providing scheduling services, providing clinical counseling and/or clinical assessment services, and providing social support network services.

In some examples, the method administering the question-based service includes establishing an intervention with the user, whereby the intervention includes at least one of a telephone call from a case manager assigned to the user or an online survey containing an inquiry for validating the predicted risk factor.

In some examples, the method includes, in accordance with a determination that the plurality of predicted risk factors includes at least one validated risk factor: determining whether the validated risk factor is an immediate risk factor, and in accordance with a determination that the valid risk factor is an immediate risk factor: generating an alert to a patient support staff and triggering administration of an immediate service associated with the immediate risk factor.

In some examples, the method includes, in response to each interaction with the user: updating the user data associated with the user, updating the plurality of predicted risk factors based on the updated user data, and calculating a new risk score based on the updated plurality of predicted risk factors.

In some examples, the method includes receiving the user data from an initial onboarding assessment of the user, categorizing, using a risk stratification model, the user into a risk class, whereby the risk stratification model utilizes common data, and calculating the first risk score based on the user data and the user's categorized risk class.

In some examples, at least one of the predicted risk factors includes a socioeconomic factor, behavioral factor, a healthcare team (“HCT”) factor, a disease factor, a drug therapy factor, and a patient financial and clinical data factor.

In some embodiments, a computer readable storage medium stores a program, and the program includes instructions, which when executed by an electronic device, cause the device to perform any of the methods described above and herein.

In some embodiments, an electronic device includes a processor, memory, and a program, and the program is stored in the memory and configured to be executed by the processor, the program including instructions for performing any of the methods of described above and herein.

In some embodiments, an electronic device includes means for performing any of the methods described above and herein.

### BRIEF DESCRIPTION OF THE FIGURES

The present application can be best understood by reference to the figures described below taken in conjunction with the accompanying drawing figures, in which like parts may be referred to by like numerals.

FIG. 1 illustrates an example distribution system including a case management system, in accordance with various embodiments of the present invention;

FIG. 2 illustrates a block diagram of example modules of an example case management system, such as the case management system of FIG. 1, in accordance with various embodiments of the present invention;

FIG. 3 illustrates a block diagram of example engines in the case management system of FIG. 2, in accordance with various embodiments of the present invention;

FIG. 4 illustrates an example method that is implemented by a case management system described herein, in accordance with various embodiments of the present invention; and

FIG. 5 illustrates an example computer system that is used to implement various embodiments of the present technology, in accordance with various embodiments of the present invention.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the present technology. Thus, the disclosed technology is not intended to be limited to the examples described herein and shown, but is to be accorded the scope consistent with the claims.

The systems and methods described herein are directed to providing healthcare solutions through various technological platforms that improve the utilization of medical articles, such as prescribed treatments and other medical goods and services. While it is well understood that proper utilization of medical articles is essential for achieving positive healthcare outcomes, several challenges still exist, such as challenges related to patient access, affordability, and adherence to the prescribed treatments. Accordingly, the systems and methods described herein are directed to addressing and mitigating such problems that hamper the successful utilization of medical articles. More specifically, the present systems and methods employ artificial technology to optimize operational efficiencies of medical article distribution and management systems, improve patient experience with such systems, and increase the impact of such systems on healthcare. For example, the present systems and methods lend to an improved and personalized patient experience, accelerate the speed to therapy, decrease abandonment rates of prescribed treatments, optimize financial assistance, and increase adherence and compliance, all of which benefits provide further advantages of increasing health outcomes, while decreasing overall healthcare costs and increasing brand loyalty to manufacturers.

As described further below, the present systems and methods are implemented as distribution systems and methods (hereinafter also referred to as “case management systems and methods”) that offer various patient support services, provider support services, and specialty pharmacy support services. Merely by way of example, such patient support services include services that facilitate reimbursement of and access to treatments, pharmacy and medical billing services, customized clinical services, risk evaluation and mitigation strategy (“REMS”) support services, case management services, patient management programs, patient assistance support, disease state training, alternative funding support, and call center services. Such provider support services can include infusion support services, durable medical equipment, revenue cycle management, clinical trial support, nursing services, clinical outcome support, and healthcare practitioner (“HCP”) office support. Further, such specialty and commercial pharmacy support services can include fee for service programs, cold chain packaging, quality management programs, data and analytics, patient journey support, therapeutic transition support, and limited distribution partnerships. With an abundant variety of services available to the patients, providers, and pharmacies, the present systems and methods identify the most effective services to be administered by applying artificial intelligence (“AI”), machine learning (“ML”), and pattern recognition through natural language processing (“NLP”) to various techniques, including predictive analytics, patient risk stratification, real-time prescription eligibility, customer relationship management, case management, and targeted patient intervention. Such techniques allow manufacturers to segment their patients and develop service programs that answer the specific needs of their populations in a faster, personalized, and more cost-effective way. This is advantageous over traditional approaches that are often hampered by inconsistency, inefficiency, limitations, and data blindness.

In certain aspects, for example, the case management system takes a multi-disciplinary approach for designing and delivering the customized support services. For instance, support services are delivered to patients and healthcare providers in the form of a clinical pharmacist, case managers, customer service representatives, field-based patient liasons, reimbursement management, and IT/data analytics. Support services are customized for the patient by the case management system based on analysis of data acquired by the system. Such data can include, but are not limited to, patient-specific data such as medical records and data collected during a welcome or onboarding call with the patient. Such data can include various combinations of data from outside sources, such as zip code, census, payor mix, demographics, CMS and private payor claims, electronic health records (“EHR”), weather, traffic, crime, PubMed, USPTO, clinical trials, medical spending, surgical spending, pharmaceutical spending, and ontologies (e.g., ICD-10, NDC, HL7, CPT). By analyzing such data, the case management system predicts potential risk factors that the patient may face in the course of treatment, such as risk factors that could impact the patient's adherence to the prescribed treatment. Specifically, medical adherence is impacted by various risks arising from socioeconomic factors (e.g., financial factors, health literacy factors, social support network factors, patient-related factors), behavioral factors, HCT factors, condition-related factors, therapy-related factors, patient financial factors, and clinical data factors (e.g., complexity of administration factors, comorbidity factors). Based on specific risk factors identified for the patient, the case management system creates the customized support service (also referred to as a “personalized intervention plan”) to mitigate the specific risk factors. Merely by way of example, such services or interventions include determining copay card eligibility, connecting with patient ambassador, providing injection training, scheduling field nurse visit, and connecting with a social worker. Such services or interventions can be provided to the patient by the patient's case manager, a field nurse, and/or any combination of the various forms of delivery listed above.

By way of further example, the patient support services contemplated herein are generally directed to initiation, onboarding, therapy access, and maintenance. For example, initiation services include central intake support (e.g., call center support, case management services, pre-dispensing patient communications) and reimbursement support (e.g., benefit investigations, prior authorization support, appeals support). Onboarding services can focus on speed to therapy (e.g., quick start programs, interim care programs, sample distribution) and financial assistance support (e.g., commercial copay access and enrollment, patient assistance program (“PAP”) enrollment and dispensing, alternate source funding solutions). Therapy access services can include clinical services (e.g., injection training support, home health support) and patient liaison services (e.g., appointment scheduling services, nurse navigators, patient ambassadors, access and data to HCP's, aligning logistical needs for appointments). Maintenance services can include adherence programs (e.g., predictive analytics, targeted engagement strategies) and patient support (e.g., psycho-social support programs, peer support programs). Each type of service can itself be customized by the case management system based on the patient's calculated risk score and risk factors. Further, an individual care plan comprising different types of services and ordering thereof can be generated based on the calculated risk score and risk factors. It will be appreciated that the case management system described herein provides various services and care plans with multiple levels available for customization based on patient-specific and treatment-specific metrics.

To demonstrate various services that can be developed and delivered using the case management system throughout a patient-product journey, an example patient-product journey is described below, illustrating how the case management system combines technology with patient services to provide an intersection that delivers optimized care throughout the patient-product journey. The journey starts with a dispensing process between the HCP and patient, during which the case management system provides services that support the HCP office with complex onboarding of the patient. Such services can be delivered by the case management system alone or in combination with a field nurse. The journey progresses to a patient support center, where the case management system can provide services such as central intake, capturing the intake by an AI engine for data synthesis, and executing a welcome call. Such services can be delivered by the case management system alone or in combination with a service hub. The journey then progresses to a benefits investigation, where the case management system provides services including patient segmentation and predictive tools that provide outputs, coordinates services that align to therapy needs and patient education, and provides resources coding assistance. Such services can be delivered by the case management system alone or in combination with a case manager and a reimbursement manager. As the journey progresses to receiving approval for the prescribed treatment, the case management system can provide services facilitating copay card enrollment, injection training as needed, PAP enrollment if required, status and data to HCP's, pre-dispensing patient communications if needed, aligning logistical needs or scheduling appointments. Such services can be delivered by the case management system alone or in combination with a case manager. Subsequently, the journey progresses to a network or specialty pharmacy, which the case management system facilitates by coordinating services that are selected and ensuring that they are provided to the patient in the various forms determined by the case management system, such as nutritionist, social workers, transportation, education, nurse outreach, etc. Such services can be delivered by the case management system alone or in combination with a field nurse and/or a case manager. Additionally, the case management system can provide services directed to dispensing shipment, providing ancillary supplies (e.g., sharps containers, vitamins, alcohol swabs), giving injection training and complete customer insight business. Such services can be delivered by the case management system alone or in combination with a field nurse. The case management system can further receive input from the field nurse including feedback after visits and interventions as they are completed, and utilizing the input to update the patient data and/or the stratification and predictive models utilized by the case management system. The journey continues with ongoing treatment and adherence, in which the case management system provides services supporting adherence and compliance, refill reminders, and executes post dispensing patient engagement. Such services can be delivered by the case management system and/or a case manager.

The patient-product journey continues throughout the prescribed treatment and beyond with future prescribed treatments, thereby requiring that services for ensuring medical adherence and preventing non-compliant behaviors are dynamically adjusted over time. The case management system described herein ensures that medical adherence is met over time by conducting multiple iterations of stratification and predictive assessments based on the patient data, which is updated after each interaction with the patient to capture various stages of the patient's journey with the prescribed treatment. For example, each patient has a unique and dynamic set of life circumstances that impact his or her ability to access, acquire, afford, and ultimately adhere to prescriptions. To provide continuous and improved healthcare solutions, the case management system identifies potential risks associated with non-adherence and non-compliant behavior throughout the course of treatment, and adapts the services selected for the patient as the patient's risk scores and risk factors change over time due to varying financial, educational, health, and/or family circumstances. As a result, the case management system described herein acknowledges that some predicted risk factors that were once mitigated may reappear as predicted risk factors in the future.

Accordingly, the case management system described herein provides various techniques for ensuring medical adherence throughout various stages of the patient-product journey. Such techniques, as described further below, include building and implementing patient risk stratification models for placing patients into risk clusters, building and implementing adherence risk predictive models for predicting patient behavior based on the patient's placement in the risk clusters, developing interventions for failure points, performing the customized interventions through patient support services teams, and continuously refining the stratification and predictive models being used, as well as updating the patient data itself based on data collected during the administration of services and interventions in order to improve medical adherence throughout the ongoing treatment. The case management system validates the predicted risk factors associated with the calculated risk scores to determine whether the potential risk factors are actual risk factors.

Further, the case management system recognizes that some predicted risk factors have greater implications than others, and thus provides an intervention plan that prioritizes services based on the level of importance of the predicted risk factor.

Still further, the case management system recognizes that in some cases, such as with untrained or short-tenured case managers, guidance is needed during interactions with patients to always take the next best step, whereby even individual care plans, risk scores, and risk factors are dynamically determined in real-time or near-real-time during administration of an intervention.

Even further, the case management system captures data from various programs, maps the data into a standard form, and resolves data inconsistencies between the programs, thereby reducing the need for patient and/or provider input (e.g., reducing survey fatigue). In some aspects, the case management system organizes the various data into an optimal data set to calculate risk scores and risk factors required by different care management programs, which can require different goals and weights and therefore yield varying risk factors. In this way, various programs and care management systems cooperatively gather data for one another, thereby minimizing the need for multiple patient and provider data requests.

In yet a further aspect, the case management system described herein provides tailored healthcare solutions at a large, scalable volume to increase patient throughput and plan participation as more effective services and increased compliance are achieved.

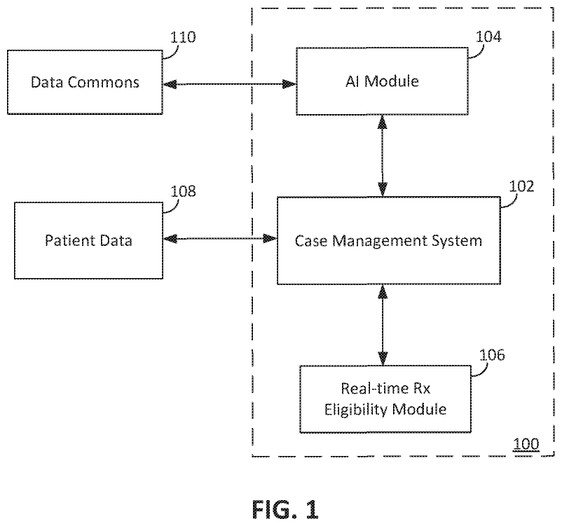

Turning now to FIG. 1, an example distribution system 100 includes a case management system 102, an AI module 104, and a real-time prescription (“Rx”) eligibility module 106. The distribution system 100 receives data from a variety of external data sources, such as patient data source 108 which may contain onboarding patient data received from an external system that is used to start a patient profile in the distribution system 100, and data commons 110. In the example shown, AI module 104 receives data from data commons 110, while the case management system 102 receives data from patient data 108. However, variations can be contemplated without departing from the invention. For example, any of the case management system 102, AI module 104, and/or the real-time Rx eligibility module 106 can receive data directly from the external sources of patient data 108 and data commons 110. Further, any of the case management system 102, AI module 104, and/or the real-time Rx eligibility module 106 can be in operative communication with one another, such that AI module 104 communicates with real-time Rx eligibility module 106. Still further, in some examples, the distribution system 100 is the case management system, and the modules shown in FIG. 1 represented by the case management system 102, AI module 104, and/or real-time Rx eligibility module 106 represent application programming interfaces (APIs) that facilitate communication between various applications and external systems, such as systems associated with manufacturers, pharmacies, providers, patients, and/or other care programs. In some examples, the case management system 102 includes capabilities of the AI module 104 and/or the real-time Rx eligibility module 106.

Merely by way of example, the API of case management system 102 interacts with the API of AI module 104 to deliver, to the AI module 104, patient onboarding data, risk factor validation, and case management patient data updates. The API of case management system 102 also receives from the AI module 104 risk scores and predicted risk factors that are calculated by the AI module 104. Further, the API of case management system 102 interacts with the API of real-time Rx eligibility module 106 to deliver patient onboarding data to the real-time Rx eligibility module. The API of the case management system 102 also receives from the real-time Rx eligibility module 106 government or commercial payor data, payor coverage, percentage of federal payout, benefits explanation and/or status, and prior authorization status. In some examples, the API of case management system 102 interacts with various APIs of various external sources providing patient data 108, and the API of AI module 104 interacts with various APIs of external sources providing data commons 110.

Still referring to FIG. 1, various modules of the distribution system 100 (and/or case management system 102) are configured to utilize a variety of data retrieved from external data sources or in some cases, collected by the distribution system 100 via input mechanisms (e.g., online questionnaires, phone calls, manual input by a case manager). The data can include program data, such as manufacturer data, payor data, and/or employer data. Further data can include disease data, ICD-10 codes, therapy-related data, drug NDCs, and/or program goals. In some examples, program goals are defined by a user, patient, case manager, provider, and/or manufacturer. The distribution system 100 manages cases based on the defined program goals, which can include goals of increased adherence, increased compliance, decreased abandonment, increased speed to access, and optimized financial assistance.

In some examples, distribution system 100 acquires and utilizes patient demographics data, patient socioeconomic data, and patient clinical data from patient data 108. Merely by way of example, patient demographics data includes name, date of birth, address, race, gender, and marital status; patient socioeconomic data includes education, income, stress level, nutrition challenges, and social support networks; patient clinical data includes the patient history and physical data, disease and comorbidities, and medication administrative data.

In some examples, distribution system 100 acquires and utilizes HCT data from data commons 110, patient data 108, and/or from HCT sources directly. The HCT data can include HCT reviews, office staff size, office or care setting or location, provider specialty, and follow-up appointments data.

In some examples, distribution system 100 acquires and utilizes payor data from data commons 110, including commercial and government payor data, plan, bin, and group numbers, primary beneficiary, drug copay and out of pocket amounts, deductible amounts, drug formulations, and prior authorization data, such as approvals, denials, appeals, and rejections.

Further, in some examples, distribution system 100 acquires and utilizes drug or manufacturer data from data commons 110 and/or manufacturer systems directly. The drug or manufacturer data can include drug name, clinical indications, dosage amount and frequency, number of refills, drug cost, manufacturer-provided plans (e.g., copay plan, quick start plan, patient assistance plan, subsidy), medicare, drug cost, medicaid, side effects and manifestation timeframe, rate of administration (e.g., oral, infused, self-infused, injected, self-injected), and tests for clinical effectiveness.

Still further, in some examples, distribution system 100 acquires and utilizes disease data from data commons 110, including ICD-10s, common comorbidities, comorbidities that impact behavior (e.g., anxiety, depression), disease stages and timing, and the impacts of external changes related to socio-economic, individual behavior, HCT, therapy, and diseases.

In some examples, the distribution system 100 acquires, manages and/or generates data related to services and interventions, such as an inventory of services that may be provided to mitigate a variety of risk factors, and/or a subset of manufacturer-selected service. Data associated with each service can include, merely by way of example, a short name, unique ID, long name, service type, questions, and actions. Each question is associated with a question ID, question constraints, and has a set of allowable answers each associated with an answer ID. Each action is associated with an action ID, action constraints, action description, action state, and action outcome. In some examples, each service selectable by the distribution system 100 corresponds to at least one question or one action. In some examples, some services have no questions or no actions.

It is noted that a variety of data can be acquired by the distribution system 100 from various external sources, and/or otherwise stored in a physical memory at the distribution system 100 or accessible to the distribution system 100 at a cloud-based memory.

Still referring to FIG. 1, in some examples, distribution system 100 utilizes the data described above to generate risk scores and predicted risk factors for each patient over time. In some cases, AI module 104 and/or case management system 102 determines whether the predicted risk factors are valid or invalid, and capture data from new interactions with the patient over time. New data can signal that there may be additional risk not originally accounted for or present. In some examples, AI module 104 and/or case management system 102 recalculates the risk scores and risk factors by accounting for the new data collected from each new interaction with the patient. Further, the distribution of services may also change over time based on the validity and statuses of the risk factors. In this way, the distribution system 100 acknowledges that patients face different challenges throughout their journey on a drug, and adapts the services provided to the patient to optimize healthcare for the patient.

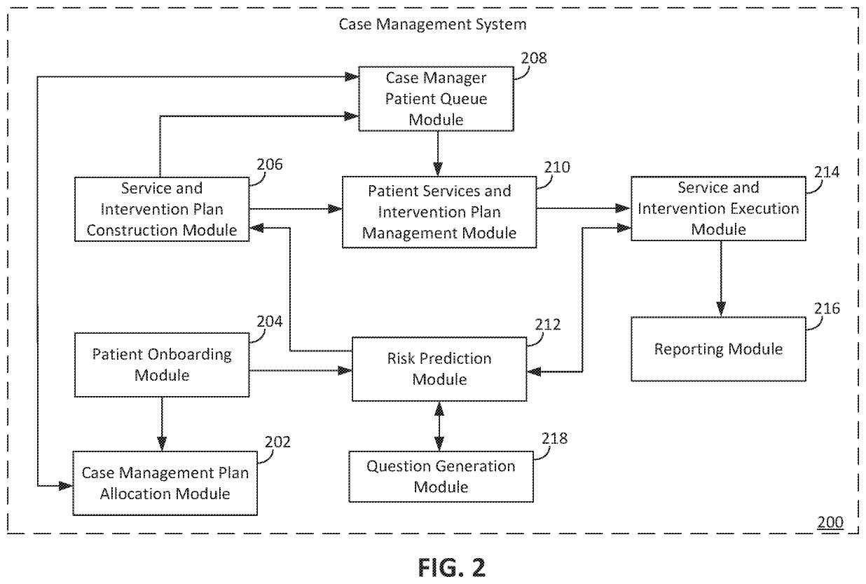

Turning now to FIG. 2, example components of a case management system 200 (e.g., which may be to the case management system 102 or the entire distribution system 100 of FIG. 1) are shown. It is contemplated that the case management system 200 provides personalized and dynamic management to improve the distribution of medical articles, and ultimately the effectiveness of healthcare solutions, by providing services that mitigate predicted risk factors. As shown in FIG. 2, the case management system 200 includes a case management plan allocation module 202, patient onboarding module 204, service and intervention plan construction module 206, case manager patient queue module 208, patient services and intervention plan management module 210, risk prediction module 212, service and intervention execution module 214, reporting module 216, and question generation module 218.

The patient onboarding module 204 captures initial intake of patient data for further data synthesis and facilitates the execution of an initial enrollment or onboarding call, such as a welcome call, enrollment call, a web enrollment, physician office staff enrollment, and/or automated phone enrollment. Data captured during the intake and initial enrollment or onboarding call is provided to the case management plan allocation module 202.

The case management plan allocation module 202 receives data related to initial intake and onboarding calls from the patient onboarding module 204. Based on the data, the case management plan allocation module 202 outputs patient segmentation and predictive tools, and helps coordinate services that align to therapy and disease state needs and patient education.

The service and intervention plan construction module 206 links predicted risk factors with one or more services. Such services can include providing an enrollment service to validate or invalidate multiple risk factors, providing access to financial assistance, providing disease or drug related education services, providing transportation services, providing scheduling services, providing clinical counseling and/or clinical assessment services, and/or providing social support network services. In some examples, such services can include facilitating copay card enrollment (e.g., for financial risk factors), connecting the user with a patient ambassador (e.g., for health literacy risk factors), providing injection training to the user (e.g., for complexity of administration risk factors), scheduling a field nurse visit with the user, and connecting the user with a social worker (e.g., for comorbidities risk factors, or situations where face-to-face interaction is preferred). In some examples, the service and intervention plan construction module 206 contains and/or otherwise accesses knowledge of confirmed or neglected risks. In some examples, the service and intervention plan construction module 206 constructs tailored services based on validated risk factors, and orders the services to provide the next best set of services to accommodate the patient's needs to meet medical adherence.

The case manager patient queue module 208 manages queues of patients in the case management system 200. In some examples, a queue of patients includes patients waiting for administration of a customized intervention plan.

The patient services and intervention plan management module 210 constructs the sequence and timing of personalized services. In some examples, the sequence and/or timing of personalized services are constructed based on the weighted importance of validated risk factors, mitigation service prerequisites, and mitigation services and/or clinical timing constraints.

The risk prediction module 212 generates risk scores (e.g., per problem and/or use case) and associated predicted risk factors.

The service and intervention execution module 214 facilitates the administration of customized services and interventions. In some examples, the service and intervention execution module 214 collects data from the patient's responses to the intervention plan or from any interaction with the patient, and feeds the data into the risk prediction module 212 to generate more questions (e.g., from question generation module 218), and more risk scores and predicted risk factors that reflect the knowledge from the new data. Example responses that can be collected from interventions can include, merely by way of example, patient feedback regarding whether a drug has been taken before, whether a drug protocol is known, and whether the drug was easy to take.

The reporting module 216 generates various patient reports, case manager reports, and manufacturer program reports.

The question generation module 218 generates questions to validate the predicted risk factors based on data collected from patient interactions.

As discussed herein, various aspects of the case management system 200 are directed to accelerating the patient's speed to therapy, decreasing the patient's abandonment rate of a medical article, optimizing financial assistance to the patient, and increasing adherence and compliance of the patient to a prescribed treatment. In some examples, the systems and methods described herein provide algorithmic predictions (e.g., via risk prediction module 212 and/or AI Module 104 of FIG. 1) by processing data and making associative connections between data, such as identifying linkages between various data elements (e.g., socioeconomic behaviors, demographics, financial payers, clinical studies, individual patient data) and utilizing the linkages in constructing models and algorithms (e.g., regression, evasion, k-nearest neighbor, random force) to make the predictions on the non-adherence risk and behaviors of an individual patient. In some examples, predictions are made (e.g., via risk prediction module 212 and/or AI Module 104 of FIG. 1) by placing the patient in a particular risk cluster and predicting failure points based at least in part on the particular risk cluster. Further, personalized intervention plans are developed (e.g., via service and intervention plan construction module 206) to address the predicted failure points, and executed through various patient support services (e.g., patient support services team) and/or case managers. The delivery of personalized intervention plans can be managed and/or otherwise coordinated by patient services and intervention plan management module 210, case management plan allocation module 202, and/or case management patient queue module 208.

Further, in some examples, the case management system 200 analyzes (e.g., via patient onboarding module 204, case management plan allocation module 202, risk prediction module 212, and/or AI module 104 of FIG. 1) common data, patient onboarding data, and/or patient input from continued interactions with the patient, and constructs patient risk stratification models to segment patients into risk clusters and develop specific interventions for each patient, while also continuously refining the stratification, interventions and/or predictive models over time. In some aspects, the case management system 200 helps manufacturers identify important clusters of patients by segmenting the patients by clusters according to government pay, Medicare/Medicaid, commercial pay, new patients, maintenance patients, age group, race, and income group.

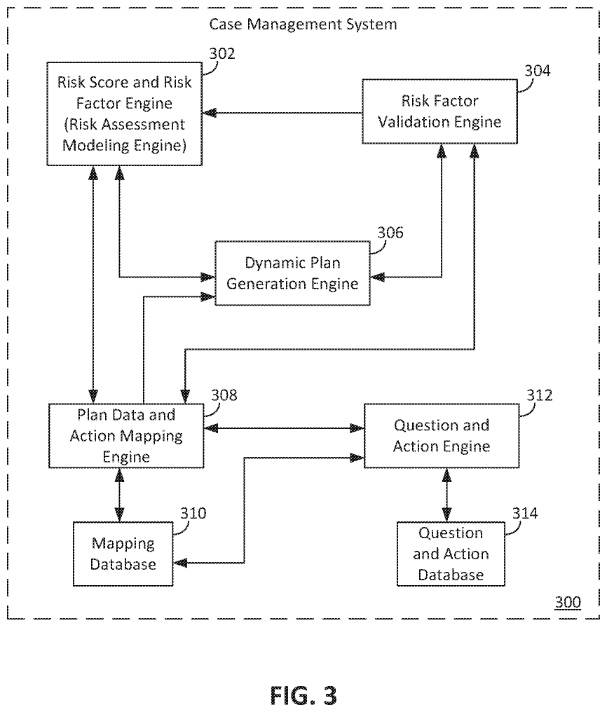

Turning now to FIG. 3, further example components of a case management system 300 (e.g., which may refer to the case management system 200 of FIG. 2, case management system 102 or the entire distribution system 100 of FIG. 1) are shown. It is contemplated that the case management system 300 generates a dynamic plan for providing one or more services or interventions directed to the mitigation of identified risk factors. As shown in FIG. 3, the case management system 300 includes a risk score and risk factor engine 302 (also referred to as a risk assessment modeling engine 302), risk factor validation engine 304, dynamic plan generation engine 306, plan data and action mapping engine 308, mapping database 310, question and action engine 312, and question and action database 314. It is contemplated that the various components shown in FIG. 3 interact with one other in various ways that are not limited by the example shown in the figure. Further, it is contemplated that case management system 300, case management system 200 of FIG. 2, and case management system 102 and/or distribution system 100 of FIG. 1 can provide functionalities and components that are similar, different, and/or interchangeable with one another without departing from the invention.

As shown in FIG. 3, in some examples, the risk assessment modeling engine 302 generates risk scores, risk factors (e.g., predicted or valid), and risk factor weights based on the type of prescribed treatment and/or based on feedback from other interventions with the patient. Risk assessment modeling engine 302 provides the risk scores, risk factors, and risk factor weighted values to the dynamic plan generation engine 306, and receives validated or invalidated risk factors from the dynamic plan generation engine 306. The risk assessment modeling engine 302 further sends plan data queries to the plan data and action mapping engine 308, and receives plan data results from the action mapping engine 308.

The risk factor validation engine 304 receives predicted risk factors from the dynamic plan generation engine 306 and sends validated or invalidated risk factor results to the dynamic plan generation engine 306.

The dynamic plan generation engine 306 determines the status of a predicted risk factor (e.g., whether it is valid or invalid) and provides the status to the risk assessment modeling engine 302, while receiving risk scores, risk factors, and risk factor weightings provided by the risk assessment modeling engine 302. The dynamic plan generation engine 306 sends potential risk factors to the risk factor validation engine 304 and receives indication from the risk factor validation engine 304 regarding valid or invalid risk factors. Further, the dynamic plan generation engine 306 receives next questions and next actions from the plan data and action mapping engine 308, and generates the dynamic plan for the patient, which when administered, data and action results can be collected and provided to the risk assessment modeling engine 302 to generate updated risk scores, risk factors, and/or risk factor weighting in real-time and/or near-real-time. It is contemplated that the dynamic plan generation engine 306 operates in real-time to constantly adjust or otherwise update an intervention or service plan for a user as new data becomes available, as risks are validated or invalidated, and as mitigation services are provided.

The plan data and action mapping engine 308 receives plan data queries from the risk assessment modeling engine 302 and sends plan data results to the risk assessment modeling engine 302. The plan data and action mapping engine 308 receives next questions and next actions from the question and action engine 312, and sends the next questions and next actions to the dynamic plan generation engine 306. Further, the plan data and action mapping engine 308 operates in conjunction with the mapping database 310, which includes libraries of questions, answers, and actions.

As discussed above, the mapping database 310 is accessible by the plan data and action mapping engine 308 and the question and action engine 312. Mapping database 310 can include public domain data, plan data, other plans data, and questions, actions, and answers.

In some examples, the plan data and action mapping engine 308 capture data from different care management programs, maps the data into a standard form, resolves data inconsistencies between the programs, and stores the data in mapping database 310. By cooperatively gathering data for multiple different case or care management programs that patients may be subscribed to, the need for numerous patient and provider data requests is mitigated or reduced. For instance, patients may be inundated with data requests from multiple case or disease managers from payers, providers, pharmacy benefit management (“PBMs”), and pharmacies, as the various entities reach out to patients to manage the patient's various diseases, medications, wellness plans, financial assistance plans, etc. Patients may grow weary of answering the same questions repeatedly when enrolling in various wellness, adherence and/or monitoring programs. In fact, multiple studies have shown that individuals tend to opt out of participating in programs due to survey fatigue. Accordingly, a benefit of the cooperative database is that patient throughput is increased as survey fatigue is reduced. Further benefits include reducing data inconsistences from patient responses, which may arise from time pressure, progression of disease, and privacy concerns.

As discussed above, it is contemplated that plan data and action mapping engine 308 and mapping database 310 support the cooperative database discussed above. Alternatively and/or additionally, other components, modules and engines can be included in the case management system 300 and in operative communication with the plan data and action mapping engine 308 and mapping database 310 to provide the cooperative database. For instance, in some examples, a registration component allows a “case/care program” to publish and subscribe to data, including data needed for the program's risk scoring and weights associated with the scoring. In some examples, a data ingestion module acquires data from registered programs and/or case management systems. In some examples, a database having a superset of timestamped risk data is provided and includes a universal set of questions each with unique ID, a set of allowable answers to each question each with a unique ID, and a collection of mappings (e.g., date of birth to age mapping, drug name to NDC mapping, disease to ICD-10 mapping). In some examples, a database of data shelf life is provided that tracks how often each piece of data should be refreshed, stores rules regarding when to refresh (e.g., a change in address may result in a request to confirm marital status), and includes various refresh triggers for transmission to selected case/care management systems based on the rules (e.g., a rule for selecting a most recent program, a rule for selecting a longest running care/case management program).

Further, in some examples, a dedicated mapping module maps individual program data to and from a canonical superset form. Merely by way of example, for data regarding alcohol use, the mapping module maps “none”, “social drinker”, and “heavy drinker” to “0-5 drinks/week”, “6-12 drinks/week”, and “>12 drinks per week”, respectively. In some examples, a rule-based module resolves inconsistencies in patient data. For example, when data from a program within a 6-month date range indicates the patient does not use alcohol, while data from another program indicates the patient is a heavy drinker, the rule-based module translates this metric to the equivalent of heavy drinker across all programs. Further, in some examples, a data transfer component allows data to be published to external programs that subscribe to the cooperative data and satisfy appropriate data usage rights (e.g., patient consent, HIPAA).

The question and action engine 312 receives data and action results from the plan data and action mapping engine 308 and/or mapping database 310, and is in operative communication with the question and action database 314. Together, the question and action engine 312 and question and action database 314 provide features for goal-driven case management system service conversations.

Specifically, a risk score and risk factor driven case management or disease management system includes a series of services that perform a number of functions, including 1) mitigating a risk through a service action, collection of data, or providing education, and 2) determining if a risk factor is an actual risk factor. For instance, risk scores are calculated based on knowledge inferred from data. For example, a system calculates a high adherence risk score when a patient's zip code is associated with low care ownership and poor public transportation system, which may indicate a potential risk factor for transportation. The case management system 300 validates the potential risk factor by an onboarding call and/or enrollment process, and continues to validate the potential risk factor multiple times during the course of a disease management program as new data and new potential risk factors are discovered based on data gathered from user interaction when new services are provided.

It is contemplated that many services are administered in the form of questions with a set of allowable responses, which are supported by the question and action engine 312 and question and action database 314. In some examples, every question-based service or risk intervention is dynamic and the only static component is the initial question. If a potential risk factor has not been validated, then the initial question attempts to validate that potential risk factor. In some cases, if the potential risk factor has been validated, then the initial question attempts to mitigate the validated risk. In some examples, question and action engine 312 and question and action database 314 generate one question-based service for each risk factor. In some examples, question and action engine 312 and question and action database 314 generate one question-based service related to multiple risk factors. For instance, instead of specific factor-related services (e.g., specific services for factors including financial assistance, transportation, injection training, comorbidities), the question and action engine 312 and question and action database 314 generates a single question-based service that always asks the next best question based on required risk factors and weighted values. In some examples, the next best question is determined to be the most important next question to drive an action or to capture additional data, whereby determination of the next best or important question is based on the weighted importance of the risk factors determined or otherwise validated or invalidated for the user. Such question-based services can be constrained by various factors, including preset time limits for each patient interaction, whereby the question and action engine 312 optimizes the question-based service to address the most important interventions within the set time period. It is contemplated that customized question-based service guides case managers in goal-driven conversations with the patient, which decreases patient burden.

For instance, in some examples, the question-based service includes one starting question. The question and action engine 312 selects the starting question with the purpose of validating a potential risk factor, such as a risk factor having a highest priority over other risk factors. If the question and action engine 312 determines that the risk factor has previously been validated, then the question and action engine 312 selects the starting question with the purpose of initiating the risk mitigation path. To generate the question-based service, the question and action engine 312 utilizes various databases that can be included in the case management system (e.g., question and action database 314 and/or mapping database 310). Such databases can include a set of data and associated weights required to calculate risk scores, a set of potential risk factors, and a superset of questions and allowable answers each with a unique ID. The question and action engine 312, alone or with the dynamic plan generation engine 306, can implement a rule-based workflow to generate a next best question in each question-based service and a rule-based workflow to organize and prioritize risk validation and risk mitigation services at each patient interaction point using the case management system 300. For each potential risk factor, the question and action engine 312, alone or with the dynamic plan generation engine 306, provides a series of question-based services with at least one question service per potential risk factor. For each question-based service, the question and action engine 312, alone or with the dynamic plan generation engine 306, provides a starting risk validation and starting risk mitigation service.

An example is provided to illustrate the question-based service described above. The example provides a question-based service for a risk factor related to transportation. Specifically, based on a set of starting data that includes a combination of zip code and census data, the risk score and risk factor engine 302 detects a low percentage of car ownership in an area where the patient and provider addresses are three miles away, a nearest pharmacy is two miles away, and the city has poor public transit. In this case, the case management system 300 determines (e.g., via risk score and risk factor engine 302) that transportation is a potential unvalidated adherence risk factor. To validate the risk factor, the question and action engine 312 and/or the dynamic plan generation engine 306 selects, from the question and action database 314, an initial question (e.g., “How many times do you drive a week?”) with a set of allowable responses (e.g., “0”, “1<5”, “>5”). The next question is dynamically generated in real-time based on the patient's response to the initial question. All responses can be captured and stored (e.g., with NLP and speech-to-text techniques, by input from the case manager administering the service, online survey). For example, if the patient responds with “0”, the dynamically-generated next question is, “Why don't you drive?” with a set of allowable responses including “Don't have a car,” “Lost my license,” “My disease/drug restricts my ability,” “I'm wheelchair bound,” and “I don't need to, I take the bus/subway.” If the patient responds to the previous question with “Lost my license,” the dynamically-generated next question is, “How many people are in your household?” Here, the question and action engine 312 and/or the dynamic plan generation engine 306 has selected a question that can validate or invalidate both the transportation-related risk factor and another potential risk factor related to social support network. If the patient's response is “>1”, a subsequent question can include, “Can your wife drive you to an appointment and pharmacy?”

In practice, the risk-validation question-generating rules or models utilized herein seek the next “most important” question in each question-based service. In some cases, the next most important question is not for the “current” program being administered, but is selected based on answers to previous questions that may be important for a different care or disease management program. For example, based on a patient's response to a food or medication allergy question, the question and action engine 312 and/or the dynamic plan generation engine 306 triggers a follow-on question due to a highly weighted (e.g., mandatory) risk factor associated with a different program. In some examples, the question and action engine 312 and/or the dynamic plan generation engine 306 utilizes data gathered from a different program to drive the question path in the current program.

Merely by way of example, in the case management system 300 of FIG. 3, the can provide a first risk factor, first risk factor weight, first risk factor validation value (e.g., True/False), and first risk factor mitigation actions. If the case management system 300 determines that a predicted risk factor is validated (e.g., True), the case management system 300 can execute a next action. If the case management system 300 determines that a predicted risk factor is not valid (e.g., False), the case management system 300 utilizes the risk factor validation engine 304 and the plan data and action mapping engine 308 to search for validation data that may be available in the mapping database 310. If no validation data is available in the mapping database 310, the case management system 300 proceeds to check if validation data is available in a different plan in the mapping database 310. If no validation data is available in a different plan, then the case management system 300 continues to generate validation questions and proceeds to utilize the dynamic plan generation engine 306, which updates the plan accordingly. A similar procedure is provided for each of the subsequent predicted risk factors, risk factor weights, risk factor validation values, and risk factor mitigation actions.

It is noted that validating predicted risk factors via the dynamically-generated and optimized question-based service enhances operability of the system and makes the system more efficient (e.g., by determining and administering services for the risk factors when they are validated, reducing the number of inputs needed to perform the operations, reducing mistakes when operating/interacting with the system or device by way of its optimized questionnaire, enhancing accuracy and effectiveness of the predictions and services provided) which, additionally, reduces power and memory usage by enabling the system to be used more quickly, efficiently, and effectively.

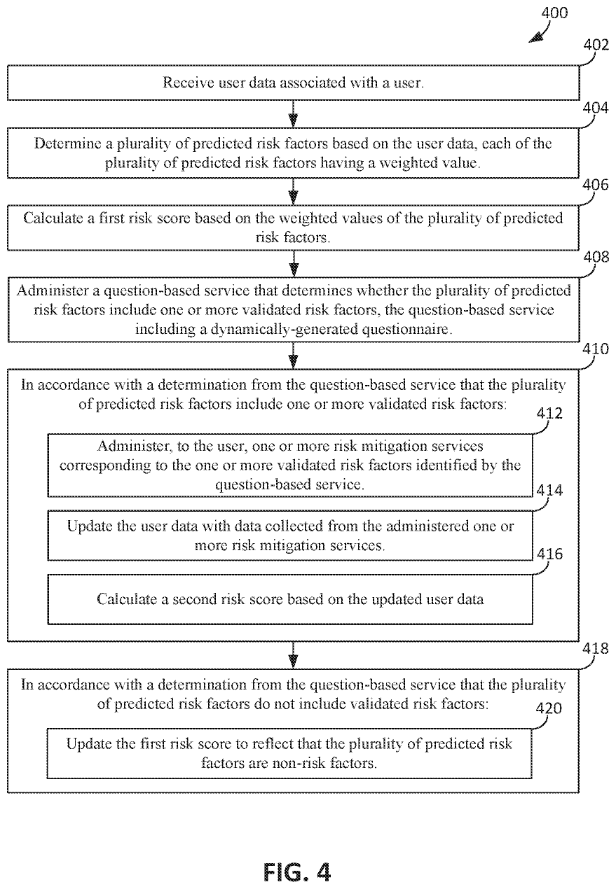

Turning now to FIG. 4, an example method 400 for distributing medical articles is provided. The method 400 can be implemented by a case management system as described above. In some examples, the method 400 is directed to increasing throughput of the case management system.

Method 400 includes receiving user data associated with a user (block 402). In some examples, the user is a patient and the user data includes medical data, behavioral data, prescribed treatment data, and other personal data. In some examples, the method 400 also receives general or common data, such as demographic data, census data, federal congressional district data, and so on. Further, in some examples, the method 400 includes updating the user data and the commons data periodically, such as with completion of every intervention, after lapse of a predetermined time period or expiration period, or when new data is detected.

In some examples, method 400 includes receiving the user data from an initial onboarding assessment of the user, such as an initial phone call to the user, whereby data from the phone call is processed using speech-to-text, NLP, or is captured and input to the case management system manually by a case manage.

In some examples, method 400 includes categorizing, using a risk stratification model, the user into a risk class, whereby the risk stratification model utilizes common data, and calculating the first risk score based on the user data and the user's categorized risk class. In some examples, the method 400 includes continuously and/or periodically modifying the risk stratification model(s) based on information from the received user input, and calculating a subsequent risk score using the modified risk stratification model. In some examples, the method 400 includes receiving drug data and disease data, and calculating the risk score based on the user data, drug data, and disease data.

The common data can include, for example, zip code data, census data, payor mix data, demographics data, National Drug Code (“NDC”) data, International Statistical Classification of Diseases (“ICD”) data, ICD-10 data, Health Level 7 (“HL7”) data, Centers for Medicare and Medicaid Services (“CMS”) data, private payor claims data, Current Procedural Terminology (“CPT”) data, electronic health records (“EHR”) data, weather data, traffic data, PubMed data, Drug Label data, United States Patent and Trademark (“USPTO”) data, clinical trials data, education data, income-related data, poverty-related data, medications availability data, comorbidities data, health insurance data, side effects data, medications profile data, competence and caregiver support data, mental health data, engagement data, and/or social network data. The user data can include demographics data, socioeconomic data, clinical history data, health care provider data, payor data, and/or medication data.

Method 400 includes determining a plurality of predicted (e.g., potential) risk factors based on the user data, where each of the plurality of predicted risk factors have a weighted value (block 404). The weighted value of each risk factor may be dependent upon the importance of the risk to certain prescribed treatments being evaluated. In some examples, the predicted risk factors are related to a medical article, such as a prescribed treatment and/or service.

In some examples, at least one of the predicted risk factors includes a socioeconomic factor (e.g., financial factor, a health literacy factor), a behavioral factor, a HCT factor, a disease factor, a drug therapy factor, or a patient financial and clinical data factor (e.g., a complexity of administration factor, a comorbidity factor).

In some examples, at least one of the plurality of predicted risk factors was previously determined to be a mitigated risk factor or a non-risk factor for the user. For example, states of risk factors can change over time such that the status of a risk factor is dynamic. Example states include potential/predicted, validated/actual, mitigated, not-yet-mitigated, non-risk. In some examples, the states of the risk factors change over time based on updating the user (e.g., patient) data after each interaction with the patient. For example, a risk for one patient may not be a risk for another even if same prescribed treatment is being provided.

Further, in some examples, the weighted value of each of the plurality of predicted risk factors for the user or patient is based on a type of prescribed treatment for the user or patient. Merely by way of example, if the user has a medical history including medications for depression or anxiety, a medical history risk factor will outweigh other predicted risk factors associated that patient being prescribed an injectable drug. For example, transportation factor is weighted 2 points, depression factor is weighted 6 points, and substance abuse factor is weighted 10 points, such that when combined, each factor has different level of importance for any problem (e.g., adherence problem, affordability problem). Specifically, the risk weighting changes from case to case.

Method 400 includes calculating a first risk score based on the weighted values of the plurality of predicted risk factors (block 406).

Method 400 includes administering a question-based service that determines whether the plurality of predicted risk factors include one or more validated (e.g., actual) risk factors, whereby the question-based service including a dynamically-generated questionnaire (block 408). In some examples, the question-based service includes a phone call between the provider and the patient. In some examples, the dynamically-generated question includes a first question that is static, and subsequent questions that are dynamically-generated during administration of the service. For example, the initial or starting question is associated with a prioritized predicted risk factor of the plurality of predicted risk factors, where the priority is determined based on weighted values of the predicted risk factors associated with the questions such that the predicted risk factors are ranked by importance. The weight of each risk factor can vary based on a type of medical or prescribed treatment. The dynamically-generated questionnaire further includes a plurality of subsequent questions (e.g., a sequence or series of questions) that are each generated (e.g., individually generated in real-time in series after each response, or generated altogether and then updated dynamically throughout the questionnaire) based on answers received to preceding questions. Such answers considered during question generation can include previous answers to previous questions in the same questionnaire, and/or historical answers to previously-administered or completed questionnaires that are stored in a database.

In method 400, the plurality of subsequent questions are further generated based on an optimization function that minimizes a total number of questions needed for the question-based service to determine whether the plurality of predicted risk factors include one or more validated risk factors. For example, the optimization function includes a dynamic decision tree, a function for generating questions based on a predetermined set of rules and/or algorithms (e.g., regression, evasion, k-nearest neighbor, random force). In some examples, the optimization function utilizes the weighted values as an indication of the next most important predicted risk factor to validate. Merely by way of example, the method 400 includes validating (or invalidating) each predicted risk factor with a goal of utilizing a lowest number of questions possible, and determining after each answer received whether more questions are needed or if the next predicted risk factor can be evaluated. In some examples, the method 400 includes determining a status (e.g., actual risk factor or non-risk factor) of each predicted risk factor dynamically in real-time and/or near-real-time, and subsequently recalculating the patient's risk score based on a changed status of a predicted risk factor.

In some examples, the dynamically-generated questionnaire is generated based on a preset time limit, where a total number of questions is constrained to a maximum number of questions allowable within the preset time limit (e.g., a maximum amount of survey time). In some examples, the dynamically-generated questionnaire is optimized for validating the predicted risk factors within the preset time limit.

In some examples, administering the question-based services includes administering the initial question to the user, whereby the initial question is associated with a plurality of predetermined allowable responses, and analyzing, in real-time, an initial answer received from the user in response to the initial question by matching the initial answer to one of the plurality of predetermined allowable responses. In some examples, method 400 includes storing data from the response and determined allowable response. Further, in some examples, method 400 includes determining, based on the matched allowable response, whether the prioritized predicted risk factor is a validated risk factor or an invalidated, non-risk factor. In some examples, method 400 includes generating, in real-time, a next question (e.g., by revising the next question or creating it) of the plurality of subsequent questions based on the matched allowable response, whereby, in accordance with a determination that the prioritized predicted risk factor is the validated risk factor or the non-risk factor, generating the next question associated with another predicted risk factor (e.g., a next priority predicted risk factor), and in accordance with a determination that the prioritized predicted risk factor is not the validated risk factor or the non-risk factor, (e.g., determining that more questions are needed to validate or invalidate the predicted risk factor) generating the next question associated with the same prioritized predicted risk factor. Further, in some examples, the method includes administering the next question to the user, whereby the next question is associated with another plurality of predetermined allowable responses for categorizing, in real-time, a next answer received from the user in response to the next question. It is noted that the set of allowable responses is dynamic in some examples—for instance, a different set of allowable responses for each case is provided for a question, even though the question remains the same across the cases.

Further, in some examples, each of the plurality of subsequent questions is administered sequentially or otherwise in series during the question-based service and the dynamically-generated questionnaire is updated in real-time in accordance with each answer received from the user. For example, the plurality of subsequent questions are generated concurrently and each question, prior to its administration, is updated (e.g., revised, replaced, dropped, made more specific, addresses multiple predicted risk factors), or unchanged, as needed, in accordance with each answer received from the user during the question-based service.

In some examples, generating or updating the dynamically-generated questionnaire includes selecting questions from a questions database, where each question corresponds to a predicted risk factor and is associated with a set of predetermined allowable responses for validating the predicted risk factor, and each question is selected based on each previously matched allowable response. In some examples, each question includes a unique ID, and each answer includes a unique ID. Merely by way of example, a question can include, “How many times do you drive a week?” and a set of allowable answers can include “0”, “1-5”, “5-10”, and “>10”. In some examples, the case management system described herein includes a database that has one question for each service and each risk factor.

Still further, in some examples, method 400 includes, while administering the question-based service, updating in real-time the first risk score in response to an answer (e.g., in response to each answer) received from the user to the dynamically-generated questionnaire, and generating or updating the dynamically-generated questionnaire based on the updated first risk score.

In some examples, the dynamically-generated questionnaire is generated based on a robustness function that minimizes errors in validating the predicted risk factors. For example, the robustness function reduces false negatives or false positives that arise in determining the status of a predicted risk factor. Merely by way of example, the robustness function can detect errors by weighting the received answers in view of the user data, prescribed treatment, and/or risk factors; weighting the probability of certain occurrences of other risks; then alerting, determining, and/or otherwise confirming whether the next question needs to be directed to confirming validation or invalidation of the predicted risk factor.

In some examples, method 400 includes determining whether the plurality of predicted risk factors includes one or more validated risk factors. In some examples, in accordance with a determination that the plurality of predicted risk factors includes at least one validated risk factor, method 400 includes determining whether the validated risk factor is an immediate risk factor (e.g., requires immediate response), and in accordance with a determination that the valid risk factor is an immediate risk factor: generating an alert to a patient support staff, and triggering administration of an immediate service associated with the immediate risk factor. In some examples, method 400 includes, in accordance with a determination that the valid risk factor is not the immediate risk factor, forgoing generating the alert and triggering the administration of the immediate service.

Method 400 includes, at block 410, in accordance with a determination from the question-based service that the plurality of predicted risk factors include one or more validated risk factors: administering, (e.g., based on a priority of validated risk factors) to the user, one or more risk mitigation services (e.g., intervention programs, question-based services) corresponding to the one or more validated risk factors identified by the question-based service (block 412), updating the user data with data collected from the administered one or more risk mitigation services (block 414), and calculating a second risk score based on the updated user data (block 416). In some examples, method 400 includes determining, in real-time, a plurality of predicted risk factors based on the updated user data, calculating the second risk score based on the newly determined plurality of predicted risk factors, and updating the validated risk factors as mitigated risk factors. In such cases, the second risk score reflects validated risk factors that have been updated to mitigated risk factors and the currently remaining validated risk factors that are not yet mitigated. In some examples, method 400 includes updating the risk score after each service is administered.

In some examples, method 400 includes, while administering each risk mitigation service of the one or more risk mitigation services, evaluating the data collected from the risk mitigation service, determining whether the validated risk factor is a mitigated risk factor based on the data collected from the risk mitigation service, and in accordance with a determination that the validated risk factor is a mitigated risk factor, calculating the second risk score based on the updated user data, whereby the second risk score is lower than the first risk score. Further, in some examples, method 400 includes updating the user data to reflect that the validated risk factor is the mitigated risk factor. In some examples, method 400 includes, in accordance with a determination that the validated risk factor is not a mitigated risk factor, determining a second risk mitigation service associated with the validated risk factor, and administering the second risk mitigation service.

In some examples, method 400 includes, in accordance with a determination that the plurality of predicted risk factors include a plurality of validated risk factors: identifying a plurality of risk mitigation services, each risk mitigation service associated with each validated risk factor, prioritizing the plurality of risk mitigation services relative to one another (e.g., based at least on the weighted value of the risk factors) to generate an intervention plan comprising a timeline for administering the plurality of risk mitigation services, and administering the plurality of services to the user in accordance with the intervention plan. In some examples, prioritizing the plurality of risk mitigation services relative to one another is based on the relative importance of the validated risk factors as indicated by their weighted values. Further, in some examples, a new risk score and/or intervention plan is generated or otherwise updated in real-time or near-real-time, during and/or after administration of each service.

In some examples, the one or more risk mitigation services includes at least one of providing an enrollment service to validate or invalidate multiple risk factors, providing access to financial assistance, providing disease or drug related education services, providing transportation services, providing scheduling services, providing clinical counseling and/or clinical assessment services, and providing social support network services.

In some examples, administering the question-based service includes establishing an intervention with the user, and the intervention includes at least one of a telephone call from a case manager assigned to the user or an online survey containing one or more inquiries for validating the predicted risk factor. The online survey can include one or more of a text message, an email, querying of an IoT device (e.g., a wearable vital signs monitor, scale, etc.), an automated phone call, an intelligent digital assistant, and/or any multiple methods that establish patient engagement. In some examples, the user is a patient or a caregiver if the patient is a child, a senior, or unable to administer their own care.

In some examples, method 400 includes, in response to each interaction (e.g., service, question-based assessment, phone call, or any interaction) with the user: updating the user data associated with the user, updating the plurality of predicted risk factors based on the updated user data, and calculating a new risk score based on the updated plurality of predicted risk factors. For instance, in some examples, method 400 includes continuously updating the user data associated with the user in real-time or near real-time in response to each new intervention with the user, whereby each new intervention includes a new user assessment, a communication with the user, or an attempted communication with the user. In some examples, method 400 includes continuously calculating a new risk score and a new predicted risk factor in real-time or near real-time in response to each new intervention with the user, where the new risk score and the new predicted risk factor are determined based on the continuously updated user data. Further, in some examples, a new or updated intervention plan is provided each time the risk score is updated or a new risk score is calculated. In some cases, method 400 includes determining, based on updated user data, that a predicted risk is mitigated or non-risk, or identifies new predicted risk factors.

Method 400 includes, at block 418, in accordance with a determination from the question-based service that the plurality of predicted risk factors do not include validated risk factors, updating the first risk score to reflect that the plurality of predicted risk factors are non-risk factors (block 420). For example, the method 400 includes updating the predicted risk factors associated with the patient profile as non-risk factors associated with the patient profile.

It is noted that the systems and methods described herein can be implemented for a variety of users, including patients and providers. For example, the distribution systems and methods disclosed herein can generate risk scores and predict risk factors for providers based on provider prescribing patterns. Merely by way of example, the systems and methods disclosed herein can identify diagnosed and undiagnosed patients for a specific disease (e.g., identify undiagnosed patients by recolonizing patterns in tests, procedures, and related diagnosis in healthcare reform (“HER”) and claims data). The systems and methods disclosed herein can locate physicians treating these patients by analyzing the data, including demographics, patient counts, specialty, drugs prescribed, drug cost, patient out-of-pocket expenses, EHR data, etc. The systems and methods described herein can receive program goals defined by the user, such as a goal to generate a risk score and risk factor relating to the likelihood of a prescriber being loyal to a branded drug and unlikely to switch to a generic or competitor, the likelihood of a prescriber being unlikely to use generics, and the likelihood of a prescriber being likely to try a new drug being launched and the prescriber's driving factors for trying the new drug (e.g., price, clinical indicators, relationship with sales rep).

Further, the systems and methods disclosed herein can be implemented for commercial payers by applying the same risk score and risk factor techniques. For example, the distribution systems and methods can receive program goals defined to increase speed of patient drug access. Risk scores and predicted risk factors can be generated based on prior authorization history of the payer, patterns in tactics that payers used to delay payment or deny prior authorization (“PA”), and mitigation factors that have been proven to be effective in overcoming payer objections. Merely by way of example, if a health insurance organization historically denies a large percentage of PAs, the techniques disclosed herein can generate a high risk score and predict risk factors including “need additional provider information,” “need additional lab test,” “need additional imaging.” By predicting such trends ahead of time, the present systems and methods mitigate the risk and eliminate any reasonable cause to deny a drug on formulary. In doing so, the present systems and methods can decrease (e.g., often by 3-4 months) the time required to get a patient on therapy.

In some examples, the distribution system and/or case management system described herein are further customizable by a user, such as a manufacturer of a prescribed treatment or therapy. For instance, the distribution system and/or case management system can include an inventory list of services that the manufacturer can choose to provide to some or all of its participants (e.g., the participants' intervention plans), or choose to opt out of. The systems and methods described herein can include receiving selection of one or more services from the inventory list that the manufacturer is willing to provide, and for each service that the manufacturer is willing to provide, further receiving rules related to providing the service, such as whether the service is mandatory for certain participants identified as having certain characteristics, mandatory for all participants regardless of individual characteristics, whether the service is associated with high, medium, and/or low levels of risk scores which can be defined by manufacturer, and/or a version or type of the service to provide. It is contemplated that the systems and methods described herein can assign services to participants based on their segmentation (e.g., risk stratification), risk scores, and/or any other characteristics, and can be customized to associate certain services with one or more risk factors or risk scores. In some examples, the systems and methods described herein receive customized services that are input by the manufacturer, and such customized services are added to the inventory list.

Merely by way of example, the inventory list includes services such as: centralized reimbursement hotline to provide basic reimbursement assistance; quick start program including vouchers and starter kits; clinical trial to commercial conversion; patient assistance programs; patient co-pay offset programs; foundation referral and application support; adverse event reporting; first-fill counseling; refill reminders and management; on-site or web-based in-services; specialty Rx coordination; patient registry; product training by field based nursing assistance; registered nurse hotline; product delivery tracking; caregiver education and support; REMS; alternative funding searches; disease management; case management; real time data reporting via portal to field; HCPs and patients; complex reimbursement assistance (e.g., coding, coordination of benefits); psychosocial support; proactive data collection for HEOR; community resource advisors; Medicaid, LIS, Part D, disability and exchange application coordination; physician appointment reminders; HQoL survey administration; field reimbursement team; e-detailing; consigned product programs; multiproduct support; replacement product; meal plans and recipe; integration with medical communication to provide standard response letters or payer information; patient finance services; sponsorship of caregiver support groups; multiple languages for forms, online and call center representatives; pharmacy price comparisons; treatment site location finder and prescription triage; compassionate use; coupon programs to provide financial benefit to government insured patients; diagnostic coordination; refill reminder call; provide welcome call; provide patient education; provide patient clinical counseling; provide injection training; treatment reminder; appointment reminder call; appointment reminder text; appointment reminder text—treatment follow-up; appointment reminder call—treatment follow-up; patient goal planning & tracking; psychoeducation—explain importance of adherence; mail product information packet to patient; mail disease information packet to patient; mail program enrollment form to patient; research aca plans for eligibility; provide Medicaid eligibility overview; refer to Medicaid; refer to mail-order pharmacy; patient and caregiver psychosocial & community support referral; patient and caregiver skilled nursing training: call support (injection or device); mail progress diary to patient; schedule appointment—injection; schedule appointment—injection follow-up; transportation support—arrange travel to physician for treatment; transportation support—arrange travel to physician for treatment follow-up; follow-up post injection call or text; research disease-state patient and family support group; find a local pharmacy; find an injector; schedule injection/med training with nurse; conduct alternative funding search; referral to patient assistance program; refer to stress management support group; refer to depression and anxiety support group; refer to alcohol dependency support group; refer to chemical dependency support group; refer to copay support—commercially insured; refer to copay support—government insured; refer to smoking cessation program; transportation support research.

In some examples, each service has a plurality of service types, such has basic, core, premium, premium plus. The manufacturer can select a service type for each service, and/or define whether a service and/or service type is included or excluded from the program services that are offered by the manufacturer. For each service and/or service type, the manufacturer can define whether the service is mandatory or not mandatory, for all or some participants. In some examples, the manufacturer defines custom segmentations for categorizing participants, such as one or more segmentations related to race and ethnicity, and/or one or more segmentations related to chronic health problems. In that case, the manufacturer defines whether to provide a selected service or service type to the customized segmentations and/or combination of segmentations. In some examples, the manufacturer specifies buckets or high, medium, and/or low risk scores, and whether to provide a selected service or service to type to such risk scores. Further, in some examples, the manufacturer associates services and/or service types to one or more risk factors, which can also be customized by the user. Such risk factors can include but are not limited to age, gender, zip code, number of medications, dosage form or drug route, dosage frequency, mental health, comorbidities, smoking status, transportation, education level, marital status, income, and social support network. In some examples, a certain service and/or service type is provided or otherwise included in an intervention plan when a manufacturer-specified combination of factors are met, such as a certain combination of the segmentations, risk score levels, and/or risk factors.

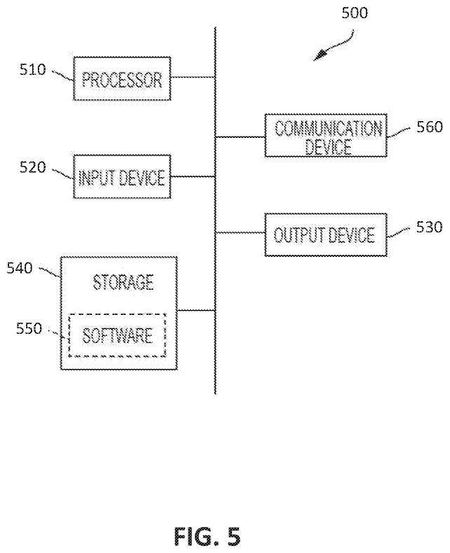

Turning now to FIG. 5, components of an example computing system or device 500 that is configured to perform any of the above-described processes and/or operations are depicted. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more of processor 510, input device 520, output device 530, storage 540, and communication device 560. Input device 520 and output device 530 can generally correspond to those described above, and can either be connectable or integrated with the computer.

Input device 520 can be any suitable device that provides input, such as a touch screen, keyboard or keypad, mouse, or voice-recognition device. Output device 530 can be any suitable device that provides output, such as a display screen, touch screen, haptics device, or speaker.

Storage 540 can be any suitable device that provides storage, such as an electrical, magnetic or optical memory including a RAM, cache, hard drive, or removable storage disk. Communication device 560 can include any suitable device capable of transmitting and receiving signals over a network, such as a network interface chip or device. The components of the computer can be connected in any suitable manner, such as via a physical bus or wirelessly.

Software 550, which can be stored in storage 540 and executed by processor 510, can include, for example, the programming that embodies the functionality of the present disclosure (e.g., as embodied in the descriptions above).

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features which may be readily separated from or combined with the features of any of the other several embodiments without departing from the scope or spirit of the various embodiments. Moreover, use of terms such as first, second, third, etc., do not necessarily denote any ordering or importance, but rather are used to distinguish one element from another.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

What is claimed is:

1. A computer-implemented method for increasing throughput of a case management system, comprising:
receiving user data associated with a user;
determining a plurality of predicted risk factors based on the user data, each of the plurality of predicted risk factors having a weighted value;
calculating a first risk score based on the weighted values of the plurality of predicted risk factors;
administering a question-based service that determines whether the plurality of predicted risk factors include a validated risk factor, the question-based service including a dynamically-generated questionnaire comprising:
an initial question associated with a prioritized predicted risk factor of the plurality of predicted risk factors; and
a plurality of subsequent questions that are each generated based on answers received to preceding questions and an optimization function that minimizes a total number of questions needed for the question-based service to determine whether the plurality of predicted risk factors include the validated risk factors
in accordance with a determination from the question-based service that the plurality of predicted risk factors include the validated risk factor:
administering, to the user, a risk mitigation service corresponding to the validated risk factor identified by the question-based service;
updating the user data with data collected from the administered risk mitigation service; and
calculating a second risk score based on the updated user data; and
in accordance with a determination from the question-based service that the plurality of predicted risk factors do not include validated risk factors:
updating the first risk score to reflect that the plurality of predicted risk factors are non-risk factors.

2. The method of claim 1, further wherein the dynamically-generated questionnaire is generated based on a preset time limit, wherein the total number of questions is constrained to a maximum number of questions allowable within the preset time limit.

3. The method of any of claims 1-2, further wherein administering the question-based services comprises:
administering the initial question to the user, wherein the initial question is associated with a plurality of predetermined allowable responses;
analyzing, in real-time, an initial answer received from the user in response to the initial question by matching the initial answer to one of the plurality of predetermined allowable responses;
based on the matched allowable response, determining whether the prioritized predicted risk factor is a validated risk factor or a non-risk factor;
generating, in real-time, a next question of the plurality of subsequent questions based on the matched allowable response, wherein:
in accordance with a determination that the prioritized predicted risk factor is the validated risk factor or the non-risk factor, generating the next question associated with another predicted risk factor, and
in accordance with a determination that the prioritized predicted risk factor is not the validated risk factor or the non-risk factor, generating the next question associated with the same prioritized predicted risk factor; and
administering the next question to the user, wherein the next question is associated with another plurality of predetermined allowable responses for categorizing, in real-time, a next answer received from the user in response to the next question.

4. The method of any of claims 1-2, further wherein each of the plurality of subsequent questions is administered sequentially during the question-based service and the dynamically-generated questionnaire is updated in real-time in accordance with each answer received from the user.

5. The method of any of claims 1-2, further wherein generating or updating the dynamically-generated questionnaire comprises selecting questions from a questions database, wherein:
each question corresponds to a predicted risk factor and is associated with a set of predetermined allowable responses for validating the predicted risk factor, and
each question is selected based on each previously matched allowable response.

6. The method of any of claims 1-2, further comprising:
while administering the question-based service, updating in real-time the first risk score in response to an answer received from the user to the dynamically-generated questionnaire; and
generating or updating the dynamically-generated questionnaire based on the updated first risk score.

7. The method of any of claims 1-2, further wherein the dynamically-generated questionnaire is generated based on a robustness function that minimizes errors in validating the predicted risk factors.

8. The method of any of claims 1-2, further wherein at least one of the plurality of predicted risk factors was previously determined to be a mitigated risk factor or a non-risk factor for the user.

9. The method of any of claims 1-2, wherein the weighted value of each of the plurality of predicted risk factors for the user is based on a type of prescribed treatment for the user.

10. The method of any of claims 1-2, further comprising:
while administering the risk mitigation service, evaluating the data collected from the risk mitigation service;
determining whether the validated risk factor is a mitigated risk factor based on the data collected from the risk mitigation service;
in accordance with a determination that the validated risk factor is a mitigated risk factor:
calculating the second risk score based on the updated user data, wherein the second risk score is lower than the first risk score; and
in accordance with a determination that the validated risk factor is not a mitigated risk factor:
determining a second risk mitigation service associated with the validated risk factor.

11. The method of any of claims 1-2, further comprising:
in accordance with a determination that the plurality of predicted risk factors include a plurality of validated risk factors:
identifying a plurality of risk mitigation services, each risk mitigation service associated with each validated risk factor;
prioritizing the plurality of risk mitigation services relative to one another to generate an intervention plan comprising a timeline for administering the plurality of risk mitigation services; and
administering the plurality of services to the user in accordance with the intervention plan.

12. The method of any of claims 1-2, wherein:
the risk mitigation service comprises at least one of providing an enrollment service to validate or invalidate multiple risk factors, providing access to financial assistance, providing disease or drug related education services, providing transportation services, providing scheduling services, providing clinical counseling and/or clinical assessment services, and providing social support network services.

13. The method of any of claims 1-2, wherein:
administering the question-based service comprises establishing an intervention with the user, further wherein the intervention comprises at least one of a telephone call from a case manager assigned to the user or an online survey containing an inquiry for validating the predicted risk factor.

14. The method of any of claims 1-2, further comprising:
in accordance with a determination that the plurality of predicted risk factors includes at least one validated risk factor:
determining whether the validated risk factor is an immediate risk factor; and
in accordance with a determination that the valid risk factor is an immediate risk factor:
generating an alert to a patient support staff; and
triggering administration of an immediate service associated with the immediate risk factor.

15. The method of any of claims 1-2, further comprising:
in response to each interaction with the user:
updating the user data associated with the user;
based on the updated user data, updating the plurality of predicted risk factors; and
calculating a new risk score based on the updated plurality of predicted risk factors.

16. The method of any of claims 1-2, further comprising:
receiving the user data from an initial onboarding assessment of the user;
categorizing, using a risk stratification model, the user into a risk class, wherein the risk stratification model utilizes common data; and
calculating the first risk score based on the user data and the user's categorized risk class.

17. The method of any of claims 1-2, wherein:
at least one of the predicted risk factors comprises a socioeconomic factor, a behavioral factor, a healthcare team factor, a disease factor, a drug therapy factor, and a patient financial and clinical data factor.

18. A computer readable storage medium storing a program, the program comprising instructions, which when executed by an electronic device, cause the device to perform any of the methods of claims 1-2.

19. An electronic device, comprising:
a processor;
memory; and
a programs, wherein the programs is stored in the memory and configured to be executed by the processor, the program including instructions for performing any of the methods of claims 1-2.

20. An electronic device, comprising:
means for performing any of the methods of claims 1-2.
